(19) United States
(12) Patent Application Publication
(10) Pub. No.: US 2021/0089820 A1
(43) Pub. Date: Mar. 25, 2021

(54) IMAGE PROCESSING APPARATUS AND STORAGE MEDIUM

(71) Applicant: Konica Minolta, Inc., Tokyo (JP)

(72) Inventors: Sumiya Nagatsuka, Tokyo (JP); Naoki Hayashi, Tokyo (JP)

(21) Appl. No.: 17/014,687

(22) Filed: Sep. 8, 2020

(30) Foreign Application Priority Data
Sep. 19, 2019 (JP) 2019-170053

(51) Int. Cl. G06K 9/62; G06T 7/00; G16H 30/40; G06F 9/50

(52) U.S. Cl. CPC G06K 9/6232; G06K 9/6224; G06T 2207/10116; G16H 30/40; G06F 9/5044; G06T 7/0012

(57) ABSTRACT

An image processing apparatus includes a hardware processor. The hardware processor is configured to determine a standard frame image in a dynamic image including a plurality of frame images obtained by imaging using radiation a process of a movement in an intentional motion by a subject. The hardware processor is configured to set a plurality of feature points on a predetermined structure in the standard frame image. The hardware processor is configured to generate position information showing a position state of the predetermined structure in the standard frame image from a relation of positions of the plurality of set feature points. The hardware processor is configured to specify in another frame image of the dynamic image positions of the plurality of set feature points and generates the position information of the predetermined structure in the another frame image.

### CROSS-REFERENCE TO RELATED APPLICATIONS

The entire disclosure of Japanese Patent Application No 2019-170053 filed on Sep. 19, 2019 is incorporated herein by reference in its entirety.

### BACKGROUND

### Technological Field

The present invention relates to an image processing apparatus and storage medium.

### Description of the Related Art

Conventionally, there is a technique to calculate a barycentric coordinate of a target positioned in a lower jaw from each one of a plurality of frame images obtained by successively irradiating an X-ray on a subject, and calculating and making a graph of a trajectory of motion of the target based on the calculated barycentric coordinate (for example, see JP 3388645).

However, for example, in the field of orthopedics, in order to objectively determine the state of a target site, it is necessary to understand the relation between the structures during the motion of the target site and the state of positions of the structure determined by the movement. Understanding only the movement of the points of the target is not enough. For example, in order to determine whether the movement of a joint is abnormal, it is necessary to at least understand the state of the positions of two or more bones (close state, angle of twist, etc.).

### SUMMARY

An object of the present invention is to obtain information to objectively understand a state of a predetermined structure during a motion.

To achieve at least one of the abovementioned objects, according to an aspect of the present invention, an image processing apparatus reflecting one aspect of the present invention includes a hardware processor, wherein, the hardware processor is configured to determine a standard frame image in a dynamic image including a plurality of frame images obtained by imaging using radiation a process of a movement in an intentional motion by a subject, the hardware processor is configured to set a plurality of feature points on a predetermined structure in the standard frame image, the hardware processor is configured to generate position information showing a position state of the predetermined structure in the standard frame image from a relation of positions of the plurality of set feature points, and the hardware processor is configured to specify in another frame image of the dynamic image positions of the plurality of set feature points and generates the position information of the predetermined structure in the another frame image.

According to another aspect of the present invention, a non-transitory computer-readable storage medium storing a program causing a computer to perform the following, determine a standard frame image in a dynamic image including a plurality of frame images obtained by imaging using radiation a process of a movement in an intentional motion by a subject, set a plurality of feature points on a predetermined structure in the standard frame image, generate position information showing a position state of the predetermined structure in the standard frame image from a relation of positions of the plurality of set feature points, and specify in another frame image of the dynamic image positions of the plurality of set feature points and generates the position information of the predetermined structure in the another frame image.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram showing an entire configuration of a dynamic analysis system according to an embodiment of the present invention;

FIG. 2 is a block diagram showing a functional configuration of an image processing apparatus shown in

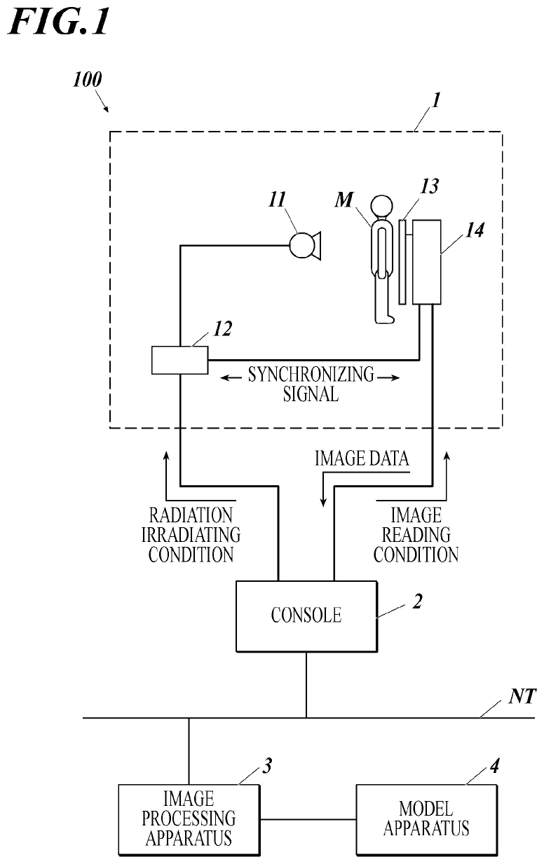

FIG. 1;

FIG. 3 is a flowchart showing an image analysis process performed by a controller of the image processing apparatus shown in FIG. 1;

FIG. 4 is a diagram to describe 3D rotation;

FIG. 5A is a diagram schematically showing a side of a lower back of a patient with spinal canal stenosis in a state standing with the spine extended;

FIG. 5B is a diagram schematically showing a side of a lower back of a patient with spinal canal stenosis in a posture tilted forward or seated;

FIG. 6 is a diagram schematically showing a side of a neck of a patient with cervical spondylotic myelopathy;

FIG. 7 is a diagram schematically showing a front view of an elbow joint;

FIG. 8A is a diagram schematically showing a swallowing movement;

FIG. 8B is a diagram schematically showing the swallowing movement;

FIG. 8C is a diagram schematically showing the swallowing movement;

FIG. 8D is a diagram schematically showing the swallowing movement;

FIG. 9 is a diagram showing an example of a feature point set in a frame image of the neck;

FIG. 10A is a graph plotting position information of cervical vertebrae generated from a dynamic image of a neck of a normal person;

FIG. 10B is a graph plotting position information of cervical vertebrae generated from a dynamic image of a neck of a patient whose cervical vertebrae is misaligned;

FIG. 11 is a diagram showing an example of an animation image of a neck; and

FIG. 12 is a diagram showing an example of a model of a neck.

### DETAILED DESCRIPTION OF EMBODIMENTS

Hereinafter, one or more embodiments of the present invention will be described in detail with reference to the drawings. However, the scope of the invention is not limited to the disclosed embodiments.

[Configuration of Dynamic Analysis System 100]

First, the configuration is described.

FIG. 1 shows an entire configuration of a dynamic analysis system 100 according to the present embodiment.

As shown in FIG. 1, the dynamic analysis system 100 includes, an imaging apparatus 1 and a console 2 connected to each other by a communication cable, etc., and a console 2 and an image processing apparatus 3 connected by a communication network NT such as a LAN (Local Area Network). A model apparatus 4 is connected to the image processing apparatus 3. The apparatuses included in the dynamic analysis system 100 conform to DICOM (Digital Image and Communications in Medicine) standard and the communication among the apparatuses is performed according to DICOM.

[Configuration of Imaging Apparatus 1]

The imaging apparatus 1 is an imager which images progress of movement of a target site by intentional motion by the subject M. The imaging apparatus 1 repeatedly irradiates pulsed radiation such as X-rays at a predetermined time interval on the target site of the subject M (pulsed irradiation) or continuously irradiates the radiation without pausing at a low dose rate on the target site of the subject M (continuous irradiation). With this, a plurality of images showing the progress of the movement of the target site is obtained. The string of images obtained by the imaging is called a dynamic image. Each of the images included in the dynamic image is called a frame image. In the embodiment described below, a series of imaging is performed by pulsed irradiation.

A radiation source 11 is positioned in a position opposite to a radiation detector 13 with a subject M in between. According to control by a radiation irradiating control apparatus 12, the radiation (X-ray) is irradiated on the target site of the subject M.

The radiation irradiating control apparatus 12 is connected to the console 2, and the radiation source 11 is controlled based on radiation irradiating conditions input from the console 2 to perform radiation imaging. The radiation irradiating conditions input from the console 2 include, for example, pulse rate, pulse width, pulse interval, number of frames imaged for each imaging, value of X-ray tube current, value of X-ray tube voltage, and additional filter type. The pulse rate is the number of times radiation is irradiated for each second, and matches with the later described frame rate. The pulse width is the radiation irradiation time for each radiation irradiation. The pulse interval is the amount of time from the start of one radiation irradiation to the start of the next radiation irradiation and matches with the later-described frame interval.

The radiation detector 13 includes a semiconductor image sensor such as a Flat Panel Detector (FPD). The FPD includes a glass substrate, for example. A plurality of detection elements (pixels) are arranged in a matrix in a predetermined position of the substrate. Radiation is irradiated from the radiation source 11 and the plurality of detection elements detect the radiation passing through at least the subject M according to its strength. The plurality of detection elements convert the detected radiation to electric signals and the signals are accumulated. Each pixel incudes a switching unit such as a Thin Film Transistor (TFT). As the FPD, there is an indirect conversion type which converts the X-ray to the electric signals with photoelectric conversion elements through a scintillator, and a direct type which directly converts the X-ray to the electric signals. Either type can be used.

The radiation detector 13 is provided opposite of the radiation source 11 with the subject M in between.

The reading control apparatus 14 is connected to the console 2. The reading control apparatus 14 controls the switching units of the pixels in the radiation detector 13 based on image reading conditions input from the console 2 and switches the reading of the electric signals accumulated in each pixel. By reading the electric signals accumulated in the radiation detector 13, the image data is obtained. This image data is the frame image. Then, the reading control apparatus 14 outputs the obtained frame image to the console 2. For example, the image reading conditions include, frame rate, frame interval, pixel size, image size (matrix size), etc. The frame rate is the number of frame images obtained in each second, and matches with the pulse rate. The frame interval is the amount of time from the start of obtaining one frame image to the start of obtaining the next frame image and matches with the pulse interval.

Here, the radiation irradiating control apparatus 12 and the reading control apparatus 14 are connected to each other. The synchronizing signals are communicated between each other and the radiation irradiating operation and the image reading operation are synchronized.

[Configuration of Console 2]

The console 2 outputs the radiation irradiating conditions and the image reading conditions to the imaging apparatus 1. In response to pressing an emitting switch, the operation of the radiation imaging and the reading operation of the radiation image by the imaging apparatus 1 are controlled and the dynamic image obtained by the imaging apparatus 1 is transmitted to the image processing apparatus 3.

[Configuration of Image Processing Apparatus 3]

For example, the image processing apparatus 3 is a computer apparatus which is used by a doctor in an orthopedics department. The image processing apparatus 3 analyzes each frame image in the dynamic image transmitted from the console 2 and generates position information showing the state of the positions of predetermined structures in a subject site (target site). The image processing apparatus 3 performs a display to describe a state of the subject site to a patient (subject M) based on the generated position information.

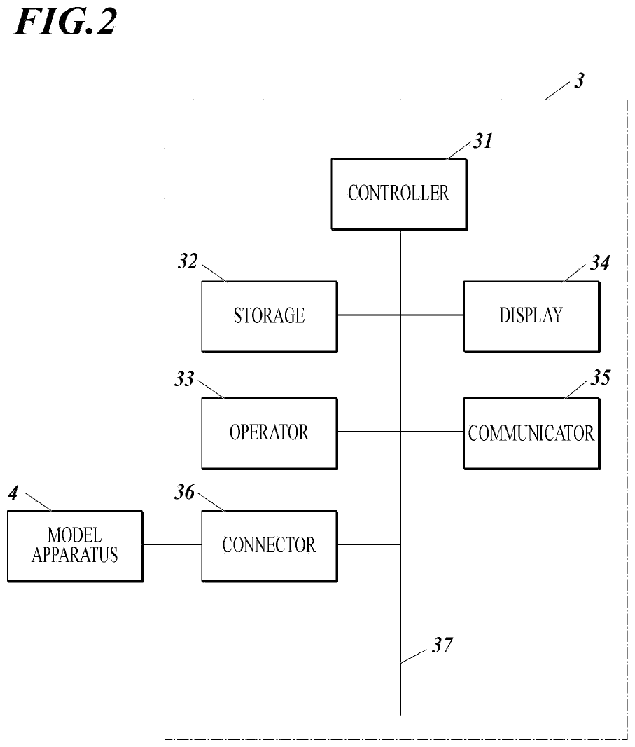

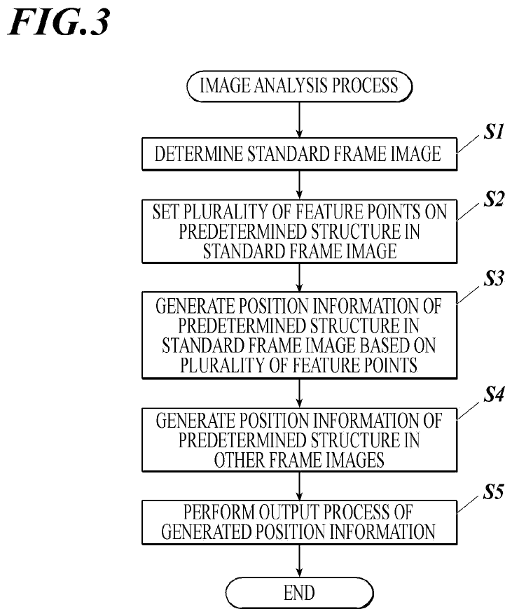

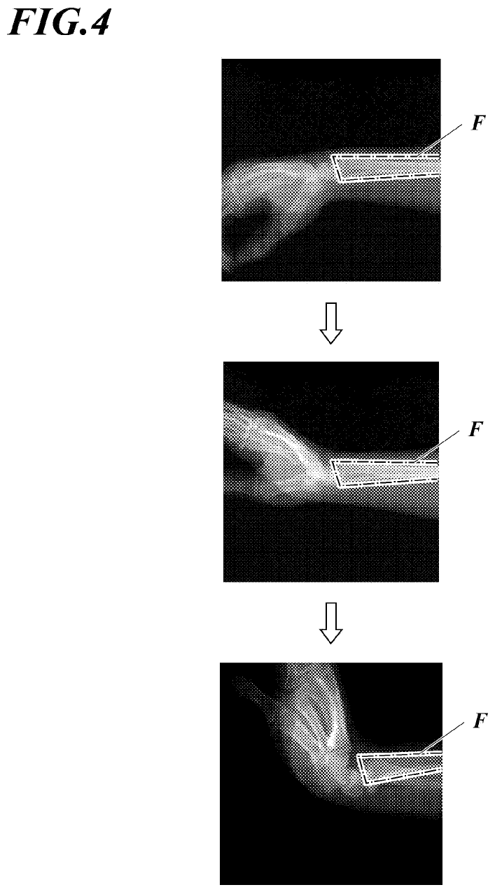

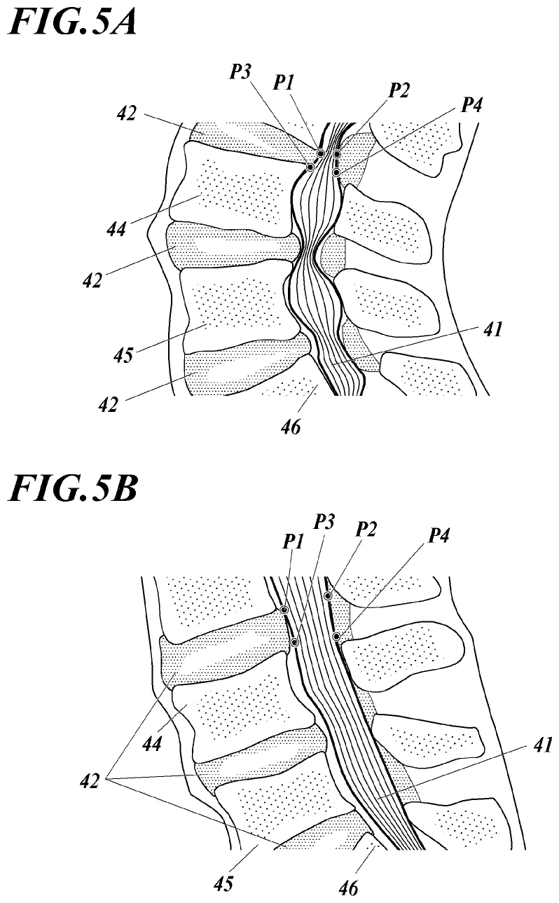

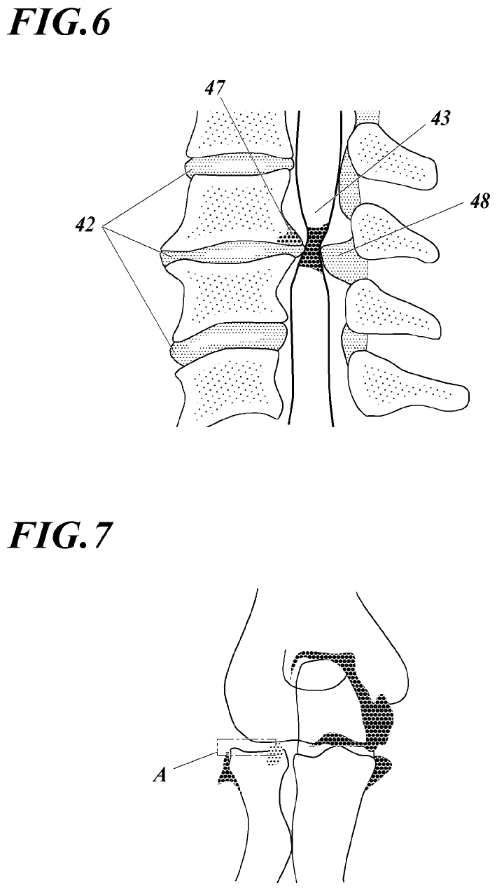

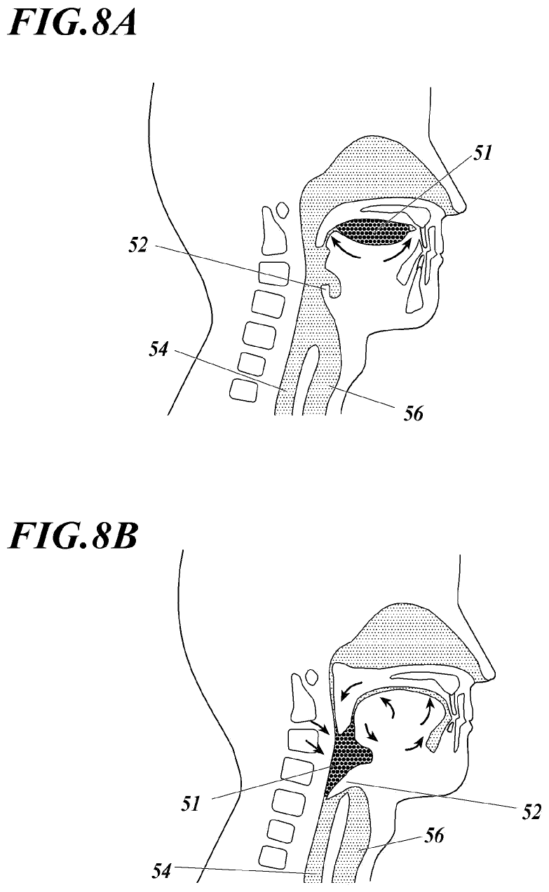

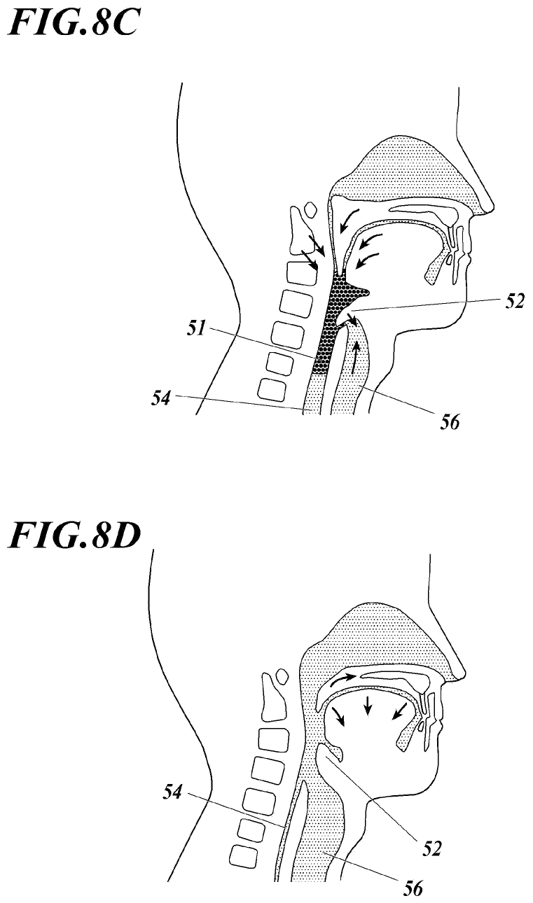

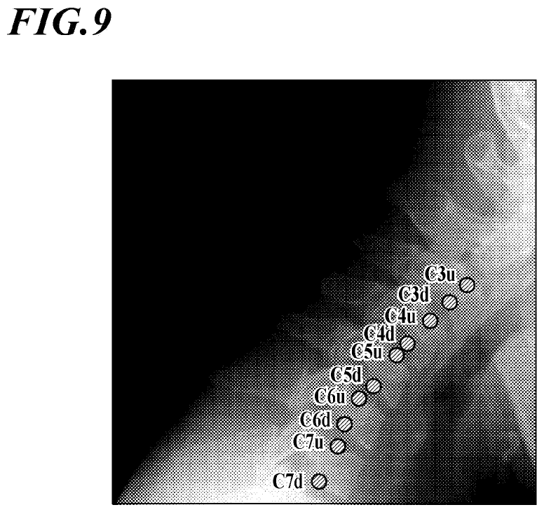

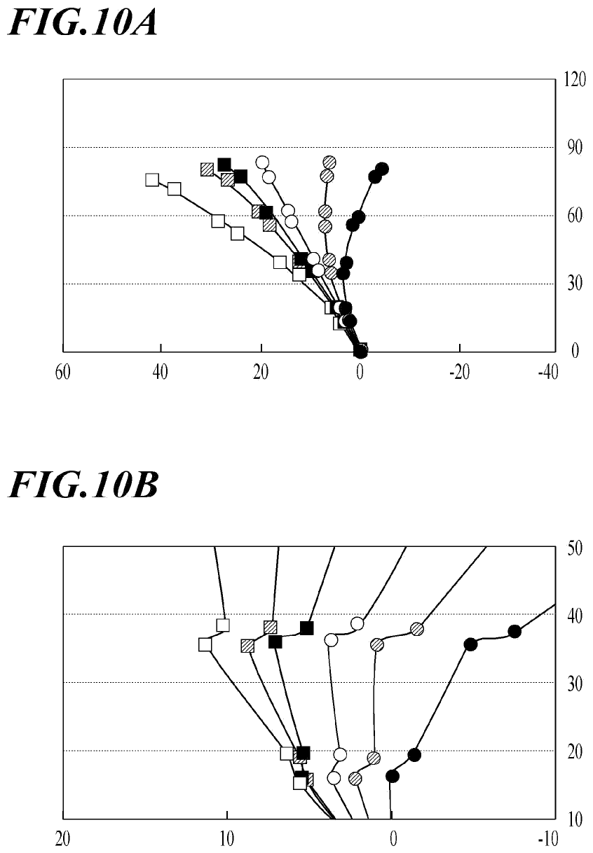

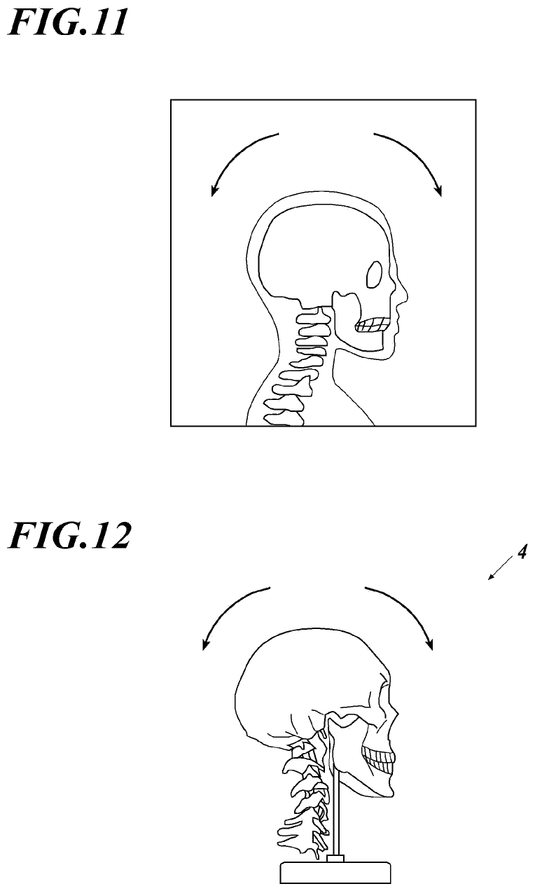

As shown in FIG. 2, the image processing apparatus 3 includes a controller 31, a storage 32, an operator 33, a display 34, a communicator 35, and a connector 36, and the above units are connected to each other by a bus 37.

The controller 31 includes a CPU and a RAM. The CPU of the controller 31 reads the system program and the various processing programs stored in the storage 32 and deploys the programs in the RAM in response to the operation on the operator 33. According to the deployed program, the controller 31 performs various processes such as a later-described image analysis process, and centrally controls the operations in each unit of the image processing apparatus 3. The controller 31 functions as a determiner, a setter, a first position information generator, a second position information generator, a difference information calculator, a determiner, a threshold value setter, an outputter, a condition setter, a display controller and a notifier.

The storage 32 includes a nonvolatile semiconductor memory or a hard disk. The storage 32 stores various programs such as programs for the controller 31 to execute the image analysis process, parameters necessary to execute the processes according to the program, and data such as results of processing. The various programs are stored in a form of a readable program code. The controller 31 sequentially executes operation according to the program code.

The operator 33 includes a keyboard provided with a cursor key, a numeric input key and various function keys, and a pointing device such as a mouse. Instruction signals input according to key operation on the keyboard and mouse operation are output to the controller 31. The operator 33 may include a touch panel on a display screen of the display 34, and in this case, the instruction signals input through the touch panel are output to the controller 31.

The display 34 includes a monitor such as a liquid crystal display (LCD) or a cathode ray tube (CRT). The display 34 performs various display according to instructions of the display signals input from the controller 31.

The communicator 35 includes a LAN adaptor, a modem, or a terminal adaptor, and controls communication of data between apparatuses connected to the communication network NT.

The connector 36 is an interface to connect with the model apparatus 4.

The model apparatus 4 includes a model of an entire human body and sites in the human body. The model apparatus 4 moves the model based on the position information output from the image processing apparatus 3 and reproduces the movement of the human body.

[Operation of Dynamic Analysis System 100]

(Imaging Operation)

Next, the operation of the dynamic analysis system 100 is described.

First, the operation of imaging is described.

An imaging operator such as a radiology technician operates the operator of the console 2 and inputs patient information (patient's name, height, weight, age, sex, etc.) of the patient (subject M) and imaging conditions (imaging site (subject site such as lower back, neck, right arm, left foot, swallowing, etc.), modality, two dimensional image or three dimensional image (3D image)). Here, modality means type of imaging apparatus 1 used in imaging (for example, standing/laying/panel (FPD) alone/long length).

When the patient information and the imaging conditions are input, the console 2 reads the radiation irradiating conditions according to the imaging site from the storage and sets the conditions in the radiation irradiating control apparatus 12 and also reads the image reading conditions according to the imaging site from the storage and sets the conditions in the reading control apparatus 14.

Here, if there is no need for a high resolution image, it is preferable to set the bit number and the frame number (frame rate) for reading the image to a low value so that the processing speed is enhanced.

The imaging operator instructs the subject M not to move the imaging site and presses the emitting switch.

Here, if the imaging of the same site for the same patient is performed in the past, it is preferable to adjust the movement to be the same cycle as the movement in the previous imaging. The adjustment of the cycle of the movement can be performed by methods such as output of a voice guiding the movement, or providing a monitor near the subject M to project images guiding the movement. Other methods can be used to adjust the movement.

In order to observe the movement which is desired to be observed in a stable state (for example, so that the movement is in an orthogonal direction with relation to the X-ray or the position of interest is most visible) imaging can be performed using jigs.

When the emitting switch is pressed, the console 2 transmits an imaging start instruction to the radiation irradiating control apparatus 12 and the reading control apparatus 14 and the imaging starts. That is, the radiation is irradiated by the radiation source 11 at a pulse interval set in the radiation irradiating control apparatus 12, and the frame image is obtained by the radiation detector 13. The frame images obtained by the imaging are sequentially input in the console 2.

When the imaging of the predetermined number of frames ends, the console 2 outputs the instruction to end imaging to the radiation irradiating control apparatus 12 and the reading control apparatus 14 and stops the imaging operation. The console 2 adds information to the input frame images such as an identification ID to identify the dynamic image, the number showing the imaged order (frame number), patient information, imaging conditions, radiation irradiating conditions, image reading conditions (for example, writes the information in the header region of the image data in a DICOM form), and transmits the above to the image processing apparatus 3.

When the imaging of the 3D image is performed, the imaging is performed from a plurality of directions.

In the static image imaging in the field of orthopedics, imaging is typically performed from four directions or six directions. Alternatively, this can be substituted by imaging dynamic images from two directions.

(Operation of Image Processing Apparatus 3)

Next, the operation of the image processing apparatus 3 is described.

When the series of frame images in the dynamic image is received from the console 2 through the communicator 35, the image analysis process shown in FIG. 3 is executed in the image processing apparatus 3 by the controller 31 in coordination with the program stored in the storage 32.

With reference to FIG. 3, the flow of the image analysis apparatus is described.

First, the controller 31 determines a standard frame image from the series of frame images in the dynamic image (step S1).

For example, the controller 31 determines a frame image satisfying conditions set in advance as the standard frame image. For example, the first frame image, the frame image in which a predetermined structure is detected for the first time (drawn), the frame image in which the position information of the predetermined structure exceeds the predetermined value or the frame image selected by the user operation (on the operator 33) is determined to be the standard frame image. If the frame image satisfying the conditions set in advance is a frame image in which a predetermined structure is detected for the first time (drawn), the controller 31 sequentially performs image recognition from the first frame image and determines the frame image in which the predetermined structure is recognized for the first time as the standard frame image. If the frame image which satisfies the conditions set in advance is a frame image in which the position information of the predetermined structure exceeds a threshold value set in advance, the controller 31 compares the position information obtained by performing the processes in later described steps S2 to S3 in order from the first frame image with the threshold value, and determines the frame image in which the threshold value is exceeded as the standard frame image.

Alternatively, the series of frame images are displayed on the display 34 and the frame image selected on the operator 33 can be determined and obtained as the standard frame image.

Next, the controller 31 recognizes the predetermined structure from the standard frame image and sets a plurality of feature points on the predetermined structure (step S2).

The predetermined structure is determined in advance by the imaging site. Alternatively, a structure can be selected as the predetermined structure from a list displayed on the display 34 by selection on the operator 33. The predetermined structure can be recognized by edge extraction, for example. The feature point on the predetermined structure can be the point specified by the operator 33 from the standard frame image displayed on the display 34. Alternatively, the frame image can be analyzed and preset positions according to the imaging site can be specified and the feature point can be set.

Next, based on the relation of the positions among the plurality of set feature points, the controller 31 generates the position information showing the state of the position of the predetermined structure in the standard frame image (step S3).

For example, the position information of the predetermined structure includes one or a combination of a plurality of the following, a straight line or curve connecting the plurality of set feature points, a surface including the plurality of feature points, an angle formed between the standard and the above straight line or surface, a distance between the plurality of feature points, and a square area of a surface formed by the plurality of feature points. The predetermined structure can be one or can be plural.

Next, the controller 31 specifies the positions of the feature points set in the standard frame image in the other frame images of the dynamic image, and generates position information in the other frame images for the structure which is the same as the predetermined structure in which the position information is generated in the standard frame image (step S4).

Next, the controller 31 performs the output process of the position information generated from the frame images (step S5) and ends the image analysis process.

In step S5, for example, the position information, the position information generated from the frame images are displayed aligned on the display 34.

Alternatively, when the position information includes values such as angle, distance, square area, etc., difference information (difference value or ratio) of the position information among a plurality of frame images (for example, between adjacent frame images) can be calculated and displayed on the display 34. With this, the frame image in which the movement of the predetermined structure became slow and the frame image in which the slow movement returned to normal can be specified. Further, it is possible to determine whether the difference information corresponding to each frame image exceeded a predetermined threshold value, and the determined result can be displayed on the display 34 together with the difference information. With this, it is possible to easily recognize whether there is an abnormality in the movement of the predetermined structure and when there is an abnormality, the posture in which there is the abnormality.

Preferably, the threshold value to determine whether the difference information shows abnormality is set by the controller 31 according to one or a combination including a plurality of the following, imaging site, modality, age, sex, and physique of patient, whether jig is used, whether an artificial material is embedded in the body, etc.

The generated position information can be displayed as a combination with the dynamic image used to generate the position information or at least one of the frame images linked to the position information.

For example, the position information corresponding to the displayed frame image is displayed switched sequentially so as to be linked with the frame images of the dynamic image switched sequentially when the dynamic image is displayed on the display 34. Alternatively, even if the display of the frame image of the dynamic image is switched sequentially, the position information (for example, the position information with the smallest (largest) value) according to the conditions set in advance is displayed continuously.

When the generated position information is displayed, according to the determination result of whether the difference information between the generated position information and the position information based on another frame image determined in advance (for example, adjacent frame image) exceeds a threshold value determined in advance, highlighted display using at least one of color, shape, size, shadow, flashing and letter type can be applied to the position information and displayed. With this, the position information which should be observed with attention can be highlighted and displayed.

The controller 31 can select and display as the frame image for initial display of the dynamic image any one of the following, the frame image in which the difference information is, lower than the threshold value set in advance, maximum, minimum, closest to the average value, or the value which changed for the first time. With this, the frame image showing characteristic movement can be initially displayed.

When the dynamic image obtained by imaging is shifted with relation to the movement direction of the frame images, preferably, the images are rotated in 3D to match with the standard movement and then displayed. For example, as shown in FIG. 4, 3D rotation is performed so that the following are constant, four points at the edge of a bone position which is not a moving portion (for example, portions shown with F in FIG. 4), intersection of diagonal lines connecting four points of the edge of the bone position, and a center of gravity of a bone square area. Alternatively, 3D rotation is performed so that the same portions in the overlapped portion of the bone which does not move becomes the largest.

Alternatively, based on the generated position information for the plurality of frame images, a graph showing the movement of the predetermined structure can be created and displayed on the display 34 (for example, see FIG. 10A and FIG. 10B). With this, the user such as a doctor can visually recognize the change over time in the state of the position of the predetermined structure.

Moreover, based on the generated position information of the plurality of frame images, an animation image reproducing the movement of the predetermined structure can be made and displayed on the display 34 (see FIG. 11). With this, the movement of the predetermined structure can be easily understood, and the accuracy of diagnosis is enhanced. Alternatively, in the department of orthopedics, the imaging result is often immediately described to the patient in the examination room. By reproducing the movement using an animation image, the patient is able to objectively understand the movement and the patient is more satisfied with the description.

When the animation image is made, the controller 31 can automatically set at least one of the following conditions according to the imaging condition at the time of imaging or the patient information, the conditions including, duplicated range of the body (limited to the site or the entire portion), duplicated size, two-dimensional or three-dimensional, color, and whether to include bone or muscle tissue. For example, the duplicated range can be set according to the imaged site. The duplicated size and color can be set by the sex, age, physique (height, weight, etc.) and the like of the subject M. Two-dimensional or three-dimensional can be set by the imaging conditions. With this, it is possible to display the animation image in a state closer to the patient.

As for a patient with artificial material such as an artificial bone in the body, the human body can be displayed in the animation image with the artificial material included. The movement of the subject with the load can be estimated and the animation image can be displayed.

Alternatively, the generated position information of the plurality of frame images can be output to the model apparatus 4 and the movement of the predetermined structure can be reproduced in the model based on the position information. With this, the movement of the predetermined structure can be easily understood and the accuracy of diagnosis can be enhanced. In the orthopedics department, the patient often receives the description of the imaging result immediately in the examination room. By reproducing the movement using a human body model, the patient is able to objectively understand how the portion is moving, and the patient is more satisfied with the description.

When the animation image is displayed or the model is moved, the display of the dynamic image can be moved linked with the above. The movement of the animation image and the model can be stopped at important frame positions. The frame image in the important position can be displayed as a still image and the moving animation image or model can be stopped when it is the movement corresponding to the frame image. The important frame image includes, the frame images with the difference information between the position information generated based on the frame image and the position information based on another frame image set in advance (for example, adjacent frame image) being, lower than the threshold value set in advance, maximum, minimum, closest to the average value, and the value which changed first.

Preferably, the format of output in step S5 is selected by the user according to operation on the operator 33.

Preferably, in steps S2 to S4, when the setting of the feature point or the generating of the position information fails, an alert by display or sound is emitted to notify information that the process failed.

The controller 31 stores the position information obtained by the above image analysis process corresponded with the dynamic image in the storage 32. Preferably, with the exception of the frame image in which the value of the position information is the maximum value, the minimum value, the average value or the value which changes first, the other frame images are stored with the resolution decreased or in a compressed state. With this, the amount of stored data can be suppressed.

Below, specific examples showing the position information calculated by the image analysis process and the output are described.

(Example of Spinal Canal)

As a condition in the spinal canal, there is spinal canal stenosis. Spinal canal stenosis is a state in which a portion of the spinal canal becomes narrow by projection of the intervertebral disk due to aging, and this compresses the nerves. With this, pain occurs. For example, as shown in FIG. 5A showing the schematic diagram of the side of the lower back when a patient with spinal canal stenosis stands and makes the spine straight, due to the increase of the swelling of the intervertebral disk 42, the spinal canal 41 is pressured and becomes narrow. On the other hand, when bent forward, or in a sitting state, as shown in FIG. 5B showing the schematic diagram of the side of the lower back, the compression of the spinal canal 41 is released, and the spinal canal 41 becomes wider. For example, the imaging apparatus 1 performs dynamic imaging from the front and the side imaging the motion from the state standing straight to the state tilted forward and the state tilted backwards. The above-described image analysis process is performed on the obtained dynamic image. With this, it is possible to determine whether the range square area or the width of the spinal canal 41 is narrow in the frame images.

For example, the controller 31 recognizes the spinal canal 41 and the intervertebral disk 42 by image recognition from the standard frame image. As shown in FIG. 5A and FIG. 5B, four points (P1 to P4) on the spinal canal 41 with the length (vertical length) of the intervertebral disk 42 as the base are set as the feature points. Next, the square area of the surface connecting the four set feature points is calculated as the position information of the spinal canal 41. Next, the positions of the features points set in the standard frame image are specified in the other frame images, and the square area surrounded by the specified features points is calculated as the position information of the spinal canal 41. Then, the position information calculated for each frame image is output by any method as described in the above step S5. With this, the doctor is able to objectively understand the change in the state of the spinal canal during the motion tilted to the front or the back (whether the spinal canal becomes narrow or not). Consequently, the doctor is able to determine whether the patient has spinal canal stenosis.

The feature points on the spinal canal 41 are not limited to the above-described P1 to P4, and the feature points can be four points based on the length of the intervertebral disk 42 between a fourth lumbar vertebra 44 and a fifth lumbar vertebra 45. Alternatively, based on the length of the plurality of intervertebral disks 42, a square area of regions formed by four feature points based on the length of the intervertebral disks 42 can be obtained, and the representative value (average value, minimum value, etc.) of the plurality of obtained square areas can be included in the position information of the spinal canal 41.

For example, the difference information of the angle of the central axis of the spinal canal 41 is calculated between the adjacent frame images, and the frame image in the section with the difference information smaller than the predetermined threshold value can be detected as the section in which there is an obstacle in the continuous movement (frame image corresponding to the motion in which the patient feels pain). For example, the center point of the region surrounded by the above-described four points based on the length of the intervertebral disk 42 between the fourth lumbar vertebra 44 and the fifth lumbar vertebra 45 and the center point of the region surrounded by the above-described four points based on the length of the intervertebral disk 42 between the fifth lumbar vertebra 45 and sacrum 46 are to be the feature points and a line connecting these points is obtained. Next, the line connecting the above two feature points in the frame image in the standing state is to be the standard line. The angle between the standard line and the line connecting the above two feature points in the frame images is obtained as the position information in the frame images. Then, the difference information between the angle calculated in the frame images and the angle calculated in the adjacent frame images is obtained. The frame image with the section in which the difference information is smaller than the predetermined threshold value is determined to be the section in which an obstacle occurred in the continuous movement (frame image corresponding to the motion in which the patient feels pain) and the result of the determination can be output together with the difference information. With this, the doctor is able to objectively understand the change in the state of the spinal canal during the motion tilted to the front and the back, and the doctor is able to determine whether the patient has spinal canal stenosis.

For example, in the range of the length (vertical length) of the intervertebral disk 42, the two points in which the width of the spinal canal 41 is narrowest (two points, front side and rear side in the side view image, left side and right side in the front view image) are set as the feature points, and the width (distance) of the set feature points is obtained as the position information of the spinal canal 41. Then, the width calculated in each frame image can be output by any method described in the above-described step S5. With this, the doctor is able to objectively understand the change in the state of the spinal canal (whether the spinal canal becomes narrow) during the motion tilted to the front and the back. Consequently, the doctor is able to determine whether the patient has spinal canal stenosis.

By comparing the square area and the width of the spinal canal 41 with the square area and the width of the spinal canal 41 at the time of the previous examination, it is possible to determine whether the state of the spinal canal 41 became worse or became better over time.

As shown in FIG. 6 showing the schematic diagram of the side of the neck, if the intervertebral disk 42 is deformed and crushed by aging in the neck, an osteophyte 47 is formed in a vertebral body and this pressures a spinal cord 43. Alternatively, if a ligament 48 becomes thick and hard, the spinal cord 43 is pressured. With this, pain and numbness occurs. This is called cervical spondylotic myelopathy. The bending and stretching motion of the cervical vertebrae can be imaged by dynamic imaging by the imaging apparatus 1. The obtained dynamic image of the cervical vertebrae can be analyzed by the analysis similar to the analysis in the dynamic image of the lower back portion as described above. With this, the square area, width or the angle can be calculated and obtained as the position information, and the position information and the difference information among the plurality of frame images can be output. Consequently, the doctor is able to objectively understand the change in the state of the spinal canal during the bending and stretching motion of the cervical vertebrae, and the doctor is able to determine whether the patient has cervical spondylotic myelopathy.

(Example of Joint)

Conditions appearing in joints include osteoarthritis in the elbow or the knee, for example.

For example, osteoarthritis is seen in the elbow of professional baseball players who continued pitching for many years. Since the elbow is used heavily, wearing of cartilage and osteophyte occur. With this, depending on the movable range, the bone is stuck or hits somewhere and causes pain. The motion of moving the elbow joint is imaged by dynamic imaging by the imaging apparatus 1, and image analysis processing is performed. The square area of the portion suspected to be the osteophyte and the space between bones are calculated, and the change in the square area when the joint is moved is output. With this, the doctor is able to objectively understand the movement of the elbow joint, and is able to determine whether the patient has osteoarthritis in the elbow. Osteoarthritis in the knee can be similarly determined from the dynamic image imaging the knee joint.

Here, since the cartilage is difficult to determine from the image, as shown in FIG. 7, the predetermined structure is surrounded by annotations A in the standard frame image of the dynamic image of the elbow joint (for example, edge of a capitulum of humerus, edge of radius head, etc.). The structure is extracted in the range surrounded by the annotation A. The feature point is set on the extracted structure and followed on the frame images. The square area of the portion surrounded by the feature points is calculated. As described above, the extracting of the structures can be performed by typical image processes such as edge detection.

(Example of Swallowing)

Swallowing is a motion of swallowing food. FIG. 8A to FIG. 8D are diagrams schematically showing correct swallowing movement. As shown in FIG. 8A, before swallowing food 51, an epiglottis 52 points upward. As shown in FIG. 8B, when food 51 is swallowed, the epiglottis 52 is lowered, a width of an esophagus 54 widens and the food 51 is guided to the esophagus 54. When the food 51 passes, the epiglottis 52 returns to pointing upwards. The dynamic image imaging the swallowing movement by the imaging apparatus 1 is processed by image analysis processing. For example, a plurality of feature points are set on the outline of the epiglottis 52 of the standard frame image, and these are followed among the frame images. The change in the shape of the curve connecting the feature points or the surface surrounded by the curve connecting the feature points capture the direction of the epiglottis 52 in the frame images. Further, two points are set opposite to each other in the esophagus 54 and the width (distance) between the points is obtained. The combination of the direction of the epiglottis 52 and the width of the esophagus 54 is output as the position information. With this, the doctor is able to objectively understand the change in the epiglottis 52 and the esophagus 54 in the swallowing movement and the relation of the above, and the doctor is able to determine whether the swallowing is normal or not. If the swallowing is not normal, it is possible to understand the reason.

For example, when swallowing is not normal, the doctor is able to determine whether the epiglottis 52 is not functioning correctly and the food 51 goes into a trachea 56 or the epiglottis 52 is pointing down but the esophagus 54 is narrow.

A width of the hypopharynx can be included in the position information.

(Example of Cervical Vertebrae)

The dynamic image showing the bending and stretching motion of the neck is imaged by the imaging apparatus 1 and the image analysis process is performed. With this, it is possible to understand the state and the movement of the cervical vertebrae.

For example, first, in the standard frame image of the dynamic image of the neck, the cervical vertebrae is recognized. As shown in C3u to C7d in FIG. 9, the feature point is set in the center of each of the upper edge and the lower edge in each cervical vertebra. Here, C3, C4, etc. in the reference numerals showing the feature point show the number of the cervical vertebrae. For example, C3 shows the third cervical vertebra and C4 shows the fourth cervical vertebra. The letters u and d added to the reference numeral showing the feature point represent upper edge, and lower edge, respectively. Instead of setting the feature point in the center of each of the upper edge and the lower edge of each cervical vertebra, the feature point can be set in the left edge or the right edge. Next, the feature points are connected by a straight line (or curve) and this is to be the position information. Next, in other frame images, the feature points are followed, and the straight line (or curve) connecting the feature points is obtained as the position information. The position information obtained from the frame images is output. For example, the position information is generated based on each frame image, plotted on the graph, and output. With this, the doctor is able to objectively understand the state of the cervical vertebrae during the bending and stretching motion.

FIG. 10A is a graph plotting the position information generated based on the frame images of the dynamic image (side) imaged during the bending and stretching motion of the normal neck. FIG. 10B extracts and enlarges a portion of the graph plotting the position information generated based on the frame images of the dynamic image imaged during the bending and stretching motion of a displaced neck. The vertical axis of the graph in FIG. 10A shows a distance upward when C7d is 0, and the horizontal axis shows the distance in the horizontal direction when C7d is 0. In the vertical axis and the horizontal axis in the graph shown in FIG. 10B, points C6d and C7u are shown in about 10 to 20 of the vertical axis, and points C5d and C6u are shown in about 40.

As shown in FIG. 10A, if the cervical vertebrae are normal, the position information in the frame images show a gentle curved shape. As shown in FIG. 10B, if there is a displacement in the cervical vertebrae, the position information in the frame images show shapes in which the lines are drastically bent at the feature points of the adjacent cervical vertebrae. It is considered that the degree of the bending at the feature points of the adjacent cervical vertebrae show the degree of the displacement of the cervical vertebrae in the location. As shown in FIG. 10B, it is possible to understand that, if there is a displacement in the cervical vertebrae, the range (angle) in which the position information moves during the bending and stretching motion is different compared to normal times.

As described above, a graph is made using the position information of the cervical vertebrae and output. With this, the doctor is able to more objectively understand the state of the cervical vertebrae during the bending and stretching motion.

If the position information in the state with the neck standing shows a straight line, it is possible to understand that the patient has a straight neck. If the position information shows a curve curved to the rear, it is possible to understand that the patient has kyphosis.

The animation image can be generated and output based on the above-described position information of the cervical vertebrae. For example, a standard animation image including the cervical vertebrae is prepared, and the center point in the upper edge and the lower edge of each cervical vertebra in the standard animation image is mapped in the position (see FIG. 9) of the center point in the upper edge and the lower edge of each cervical vertebra according to the position information generated based on the frame images. With this, the animation image is made and output. FIG. 11 shows an example of the animation image. With this, since the animation image reproducing the movement of the cervical vertebrae during the bending and stretching movement can be output, the movement of the cervical vertebrae can be easily understood, and the diagnosis accuracy can be enhanced. Moreover, if the movement is reproduced in the created animation image, the patient is able to objectively understand how the portion is moving when the patient receives the description, and the patient is more satisfied with the description.

The position information of the cervical vertebrae can be output to the model apparatus 4. The movement of the cervical vertebrae during the bending and stretching motion can be reproduced in the model apparatus 4. For example, in the model apparatus 4, the center point in the upper edge and the lower edge of each cervical vertebrae of the model apparatus 4 is moved to the position of the center point in the upper edge and the lower edge of each cervical vertebrae in the position information corresponding to the output frame image, and the movement of the cervical vertebrae of the patient is reproduced. FIG. 12 shows an example of the model apparatus 4. With this, since the movement of the cervical vertebrae can be reproduced, the movement of the cervical vertebrae can be easily understood, and the diagnosis accuracy can be enhanced. Moreover, if the movement is reproduced in the model apparatus 4, the patient is able to objectively understand how the portion is moving when the patient receives the description, and the patient is more satisfied with the description.

As described above, according to the image processing apparatus 3, the controller 31 determines a standard frame image in a dynamic image including a plurality of frame images obtained by radiation imaging of a process in a movement of an intentional motion by the subject. The controller 31 sets a plurality of feature points on a predetermined structure in the standard frame image. The controller 31 generates position information showing the position state of the predetermined structure in the standard frame image from the relation of the positions of the plurality of set feature points. Then, the position of the plurality of feature points set in the standard frame image is specified in the other frame images of the dynamic image. With this, the position information of the predetermined structure is generated in the other frame images.

In the field of orthopedics, in order to objectively determine the state of the target site, there is meaning in understanding the relation among the structures during the motion of the target site and the state of the position of the structure determined by the motion. According to the controller 31, the position information showing the position state of the predetermined structures is generated from the frame images in the dynamic image. Therefore, it is possible to provide information to objectively understand the state of the predetermined structure during the motion of the subject site.

The difference information of the position information between plurality of frame images such as adjacent frame images can be calculated, to be able to understand the change in the movement of the predetermined structure in the frame images. With this, it is possible to specify the posture in which the movement becomes bad.

By determining whether the difference information exceeds a predetermined threshold value, it is possible to determine whether there is an abnormality in the movement of the predetermined structure. Moreover, it is possible to perform determination of the abnormality based on the threshold value according to the capturing condition and the subject by setting a threshold value according to one or a combination of a plurality of the following such as imaged site when imaging the dynamic image, modality, age of the subject, sex, physique, whether the jig is used and whether there is an artificial material in the body.

The graph or the animation image is created and output showing the movement of the predetermined structure based on the position information generated in a plurality of frame images or position information is output to the external model apparatus linked with the position information. With this, the movement of the structure can be easily understood and the diagnosis accuracy can be enhanced. By showing the graph, animation image, model, etc. to the patient, the patient is able to objectively understand how the portion is moving, and the patient is more satisfied with the description.

At least one of the following conditions for making the animation image is set according to the imaging condition at the time of imaging or the patient information, the conditions including, duplicated range of the human body, duplicated size, two-dimensional or three-dimensional, color, and whether bone or muscle tissue exist. With this, the animation image with the human body close to the sex, age, physique, etc. of the patient can be output.

With the exception of the frame images in which the value of the position information include the maximum value, minimum value, average value or the value which changes first among the values of the position information calculated from the plurality of frame images, the other frame images are stored with the resolution reduced or in a compressed state. With this, the data amount can be reduced when the dynamic image is stored.

Linked with each frame image of the dynamic image displayed on the display 34 switching sequentially, the position information corresponding to the displayed frame image is displayed switched sequentially With this, it is possible to display the frame image and the corresponding position information associated with each other. Alternatively, even if each frame image of the dynamic image is switched, the position information according to a preset condition can be displayed continuously. With this, the important position information can be displayed with the dynamic image.

In response to the determination result showing whether the difference information between the position information and the position information based on the other frame image determined in advance exceeds the threshold value set in advance, the highlighted display is applied to the displayed position information using at least one of the following, such as color, shape, size, shadow, flashing, and character type. With this, the user is able to intuitively understand the position information with the abnormality.

When the difference information between the position information and the position information based on another frame image set in advance is lower than the threshold value set in advance, maximum, minimum, closest to the average value or changed first, the frame image is displayed as the frame image for the initial display of the dynamic image. With this, it is possible to initially display the frame image including the characteristic movement or the frame image including the average movement.

When the setting of the feature point or the generating of the position information fails, by notifying that the process failed, the user is able to understand that the setting of the feature point or the generating of the position information failed.

The description according to the present embodiment is one example of the suitable dynamic analysis system according to the present invention, and the present invention is not limited to the above.

For example, when the dynamic image which images the subject site by dynamic imaging is displayed in the field of orthopedics, the following methods are also effective for diagnosis and description to the patient.
Displaying the imaged dynamic image with the dynamic image imaged in the past aligned with each other in a state with the cycle matched.
Displaying the imaged dynamic image aligned with the X-ray still image in a predetermined state.
Displaying the imaged dynamic image aligned with the image showing the normal movement of the imaged site.
Displaying the video imaging the imaging site with a video camera aligned with the dynamic image by X-ray.
Displaying the difference from the previously imaged dynamic image on the image.
Displaying the 3D dynamic image generated by imaging from a plurality of directions.
Stopping the display of the dynamic image at the frame image with the largest difference from the frame image corresponding to the dynamic image previously imaged.
Stopping the dynamic image at the frame image with the unnatural movement (for example, the frame image in which the difference value of the position information between the adjacent frame image exceeds the threshold value set in advance).

The 3D printer is connected to the image processing apparatus 3. The image data of the 3D image generated by imaging the subject site in a predetermined state from a plurality of directions is transmitted to the 3D printer. The model is generated based on the image data in the 3D printer. Such method is effective for the description for the patient. For example, the 3D image data with the width of the spinal canal in the narrowest state is output to the 3D printer. The model of the subject in such state is created with the 3D printer. With this, the posture in which the spinal canal is the narrowest and in which pain occurs can be shown as a model to the patient.

For example, according to the above description, a hard disk or a semiconductor non-volatile memory is used as the computer-readable medium including the program of the present invention, but the present invention is not limited to the above. A portable storage medium such as a CD-ROM can be applied as the computer-readable medium. A carrier wave can be applied as the medium to provide data of the program regarding the present invention through a communication line.

The detailed configuration and the detailed operation of each device included in the dynamic analysis system 100 can be suitably modified without leaving the scope of the present invention.

For example, various processing functions of the image processing apparatus 3 can be included in the console 2 or the image storing apparatus such as Picture Archiving and Communication System (PACS) (not shown). Alternatively, the processes performed in the image processing apparatus 3 can be performed in the cloud server provided in a cloud environment, and the generated position information and difference information can be distributed to other servers as the cloud service.

Although embodiments of the present invention have been described and illustrated in detail, the disclosed embodiments are made for purposes of illustration and example only and not limitation. The scope of the present invention should be interpreted by terms of the appended claims

What is claimed is:

1. An image processing apparatus comprising:
a hardware processor, wherein,
the hardware processor is configured to determine a standard frame image in a dynamic image including a plurality of frame images obtained by imaging using radiation a process of a movement in an intentional motion by a subject,
the hardware processor is configured to set a plurality of feature points on a predetermined structure in the standard frame image,
the hardware processor is configured to generate position information showing a position state of the predetermined structure in the standard frame image from a relation of positions of the plurality of set feature points, and
the hardware processor is configured to specify in another frame image of the dynamic image positions of the plurality of set feature points and generates the position information of the predetermined structure in the another frame image.

2. The image processing apparatus according to claim 1, wherein the position information includes one or a combination of a plurality of the following, straight line or curve connecting the plurality of feature points, surface formed from the plurality of feature points, angle formed between the straight line or the surface and a standard, a distance between the plurality of feature points, and a square area of the surface formed by the plurality of feature points.

3. The image processing apparatus according to claim 1, wherein the hardware processor is configured to calculate difference information of the position information among a plurality of frame images.

4. The image processing apparatus according to claim 3, wherein the hardware processor is configured to determine whether the difference information exceeds a threshold value set in advance.

5. The image processing apparatus according to claim 4, wherein the hardware processor is configured to set the threshold value according to one or a combination of a plurality of the following, an imaging site when the dynamic image is imaged, modality, age, sex, physique of the subject, whether a jig is used, and whether there is artificial material inside a body.

6. The image processing apparatus according to claim 1, wherein the hardware processor is configured to create a graph or an animation image showing a movement of the predetermined structure based on the position information generated in the plurality of frame images and output the graph or the animation image, or to output the position information to an external model apparatus linked with the position information.

7. The image processing apparatus according to claim 6, wherein the hardware processor is configured to set at least one of the following conditions including duplicated range of a human body, duplicated size, two-dimensional or three-dimensional, color, and whether bone or muscle tissue is included according to an imaging condition during imaging or patient information when the animation image is created.

8. The image processing apparatus according to claim 1, further comprising a storage which stores the position information.

9. The image processing apparatus according to claim 8, wherein, with an exception of frame images in which the value of the position information is a maximum value, a minimum value, an average value, or a value which changes first, among position information calculated from the plurality of frame images, the storage stores the other frame images with resolution reduced or in a compressed state.

10. The image processing apparatus according to claim 1, wherein the hardware processor is configured to display on a display the position information alone or to display on the display the position information combined with at least one of the dynamic image or the frame image corresponding to the position information.

11. The image processing apparatus according to claim 10, wherein the hardware processor is configured to display on the display the position information corresponding to the displayed frame image to sequentially switch linked with an operation of each frame image in the dynamic image being switched sequentially, or to continuously display on the display the position information according to a condition set in advance even if each frame image in the dynamic image is switched.

12. The image processing apparatus according to claim 10, wherein the hardware processor is configured to display the position information applying display of highlight using at least one of the following including, color, shape, size, shadow, flashing, or character type according to a determination result that difference information between the position information and the position information based on the another frame image set in advance exceeds a threshold value set in advance.

13. The image processing apparatus according to claim 10, wherein the hardware processor is configured to display as a frame image for initial display of the dynamic image any one of the following among the plurality of frame images, the frame image which includes difference information between position information based on the frame image and position information based on the another frame image set in advance being lower than a threshold value set in advance, the frame image which includes the difference information being maximum, the frame image which includes the difference information being minimum, the frame image which includes the difference information being closest to an average value, or the frame image which includes the difference information being first to change.

14. The image processing apparatus according to claim 1, wherein the hardware processor is configured to notify failure when setting of the feature point or generating of the position information fails.

15. The image processing apparatus according to claim 1, wherein the hardware processor is configured to determine a first frame image, a frame image in which the predetermined structure is detected for the first time, a frame image in which the position information of the predetermined structure exceeds a predetermined value or a frame image selected by user operation as the standard frame image.

16. The image processing apparatus according to claim 1, wherein the dynamic image is a dynamic image which images the following with radiation, a motion moving a lower back, a motion moving a neck, a motion moving a joint or a swallowing motion.

17. A non-transitory computer-readable storage medium storing a program causing a computer to perform the following,
determine a standard frame image in a dynamic image including a plurality of frame images obtained by imaging using radiation a process of a movement in an intentional motion by a subject,
set a plurality of feature points on a predetermined structure in the standard frame image,
generate position information showing a position state of the predetermined structure in the standard frame image from a relation of positions of the plurality of set feature points, and
specify in another frame image of the dynamic image positions of the plurality of set feature points and generates the position information of the predetermined structure in the another frame image.
